(19) United States
(12) Patent Application Publication
(10) Pub. No.: US 2016/0135748 A1
(43) Pub. Date: May 19, 2016

(54) Inflatable Electroencephalogram Measuring Device

(71) Applicant: Brain Rhythm Inc.

(72) Inventors: Chin-Teng LIN, Jhubei City (TW); Shao-Wei LU, Hsinchu City (TW); Shi-Wen CHANG, Hsinchu City (TW); Yi-Hsin YU, Hsinchu City (TW)

(21) Appl. No.: 14/925,607

(22) Filed: Oct. 28, 2015

(30) Foreign Application Priority Data
Nov. 19, 2014 (TW) 103139989

(51) Int. Cl. A61B 5/00; A61B 5/0478

(52) U.S. Cl. CPC A61B 5/6843; A61B 5/0478; A61B 5/6803; A61B 2562/0247

(57) ABSTRACT

An inflatable electroencephalogram measuring device comprises an inflatable helmet applicable for an user's head and at least one measuring electrode. A plurality of supporting strips are disposed within the inflatable helmet and the at least one measuring electrode is well distributed upon the supporting strips. When the inflatable helmet is inflated, the measuring electrode accordingly moves to make contact with the use's head so as to measure an electroencephalogram signal. By designing the inflatable electroencephalogram measuring device with an inflatable helmet, the proposed invention is widely utilized for measuring various users with a variety of head sizes, and thus is characterized with a potential for becoming an important tool of medical measurement.

This application claims priority for Taiwan patent application no. 103139989 filed on Nov. 19, 2014, the content of which is incorporated by reference in its entirely.

### BACKGROUND OF THE INVENTION

1. Field of the Invention

The present invention is related to an electroencephalogram measuring device, and more particularly to an electroencephalogram measuring device which provides an inflatable helmet to wear, and by inflating the inflatable helmet it is widely utilized for measuring various users with a variety of head sizes.

2. Description of the Prior Art

In general, human brains are constantly producing electrical impulses during every second, every day no matter who you are and what you are doing even in deep sleep. These electrical impulses are so far known as “brain waves”. Generally, these brain waves are classified as β-waves (consciousness), α-waves (between consciousness and sub-consciousness), θ-waves (sub-consciousness) and δ-waves (unconsciousness) according to their different frequencies. Since these various waves constitute inner behaviors, outer behaviors and learning behaviors of a person, modern technology has been able to detect and show human-brain waves on monitor through a variety of measuring devices so as to provide further analyses for future developments and researches.

Nowadays, there are mostly two types of technologies to measure brain waves: one is electroencephalogram (EEG) for measuring pallium electrical currents, which are generated outside the brain cells and formed by voltage drops between cells to cells; and the other is magnetoencephalographic (MEG) which is performed based on Faradays' Law, wherein when the brain nerves are activated, the generated electrical signals induce magnetic field to produce magnetic flux changed so as to measure the MEG signal.

However, in practical measuring process, a testee must wear a specific measuring device over his or her head so as to record the brain waves generated in a certain period for analyzing its changes. Nevertheless, it should be noticed that for different testees, their head sizes also varies a lot, which becomes a main issue to overcome since most of the measuring devices today are not designed to be fully applicable for all head sizes. As such, the conventional measuring devices developed so far are thus being limited and cannot be widely used in the market nowadays.

As a result, on account of all, it should be obvious that there is indeed an urgent need for the professionals in the field for a new inflatable electroencephalogram measuring device to be developed that can be widely utilized to various users having a variety of head sizes so as to solve the above-mentioned problems occurring in the prior art.

### SUMMARY OF THE INVENTION

In order to overcome the above-mentioned disadvantages, one major objective in accordance with the present invention is provided for a novel and creative inflatable electroencephalogram measuring device. By employing the novel inflatable electroencephalogram measuring device, it can be utilized not only to successfully solve the conventional issues occurring in the prior art, but also to make it possible to perform instantaneous and extended EEG measurements. Besides, the measured EEG signals in the proposed design are also characterized with high accuracy and precision, and thus have the potential for becoming an important tool of medical and researches developments in the future.

Another objective in accordance with the present invention is provided for a novel inflatable electroencephalogram measuring device, wherein the EEG measuring device comprises an inflatable helmet, and by inflating the helmet, at least one measuring electrode disposed inside the helmet can be accordingly induced to move closed to the testee's head. Therefore, the proposed inflatable electroencephalogram measuring device is believed to fit all testees having various head sizes.

And yet another objective in accordance with the present invention is provided for a novel inflatable electroencephalogram measuring device, which provides not only extraordinary measuring properties but also excellent adhesion to the testee's head. As such, the proposed inflatable electroencephalogram measuring device is convenient to use, advantageous of low production complexity and less cost consumption.

For achieving the above mentioned objectives, the present invention provides an inflatable electroencephalogram measuring device, which comprises an inflatable helmet and at least one measuring electrode. The inflatable helmet is used for a testee to wear over the head and comprises an inflatable body and a plurality of supporting strips. The supporting strips are disposed inside the inflatable body, and the measuring electrode is disposed upon the supporting strips. As such, when the inflatable helmet is inflated, the measuring electrode is induced to make contact with the testee's head so as to measure EEG signals. In the embodiment, the inflatable electroencephalogram measuring device is considered to be an independent air bag designed in a helmet shape, and the measuring electrode is configured in the inner space of the air bag. As a result, by inflating the air bag, the inflation oppresses the measuring electrode such that the measuring electrode effectively contacts with the testee's head.

Furthermore, according to one embodiment of the present invention, the inflatable helmet further comprises an inlet, which is disposed on a side of the inflatable body. The inflation toward the helmet can be done through air being input to the inlet.

Moreover, the supporting strips of the present invention are made of flexible materials, such that each supporting strip is bendable. By bending the supporting strips, it absorbs the stress generated by the moving measuring electrode. As such, the testee may enjoy better comfort when wearing the measuring device and the measuring device itself is much more delicate and soft than ever.

And yet, according to another embodiment of the present invention, the inflatable helmet can also be designed as an air bag embedded in a hard case. Under such circumstance, the proposed inflatable electroencephalogram measuring device further comprises a housing, and the inflatable helmet is disposed in the inner space of the housing for the testee to wear and measure the EEG signals.

Therefore, it is apparent that the inflatable electroencephalogram measuring device of the present invention merely needs to use an inflatable helmet and by inflating the helmet, the measuring device is applicable to all users having different head sizes. Compared to the prior arts, the present invention is much easier to fabricate, thereby reducing its production structural cost. Furthermore, controlling the level of inflation to the helmet also makes it suitable for all head sizes and meanwhile still maintains excellent measuring properties.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings:

FIG. 1 shows a three-dimensional schematic drawing of the inflatable electroencephalogram measuring device in accordance with one embodiment of the present invention.

FIG. 2 shows an inner drawing of the inflatable electroencephalogram measuring device in accordance with one embodiment of the present invention.

FIG. 3 shows a schematic drawing of the inflatable body in accordance with one embodiment of the present invention.

FIG. 4 shows a schematic drawing of the inflatable body which comprises more than one chamber in accordance with another embodiment of the present invention.

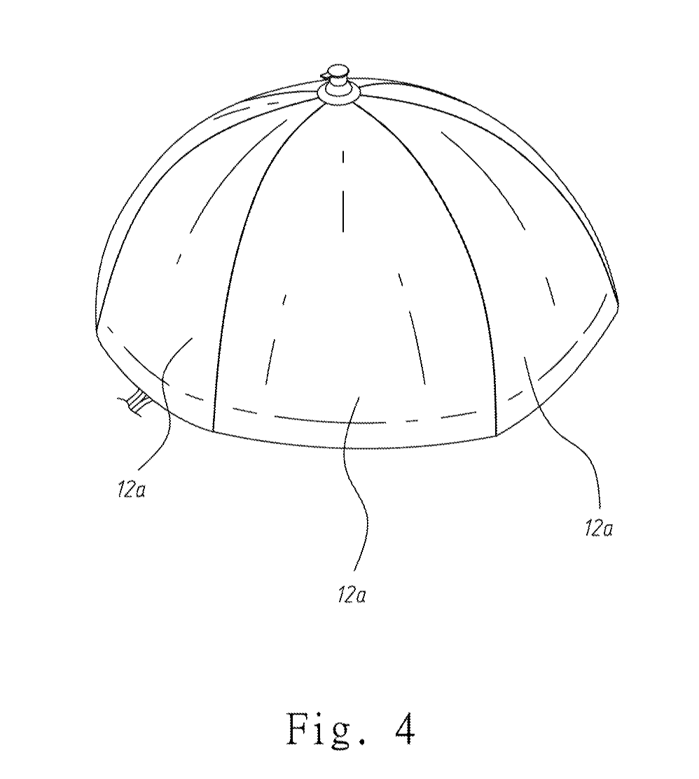

FIG. 5 shows a schematic drawing of the inflatable electroencephalogram measuring device which comprises a housing in accordance with another embodiment of the present invention.

FIG. 6 shows a side drawing of a testee wearing the inflatable electroencephalogram measuring device shown in FIG. 5.

FIG. 7 shows a schematic drawing of the inflatable electroencephalogram measuring device shown in FIG. 5 which has been inflated.

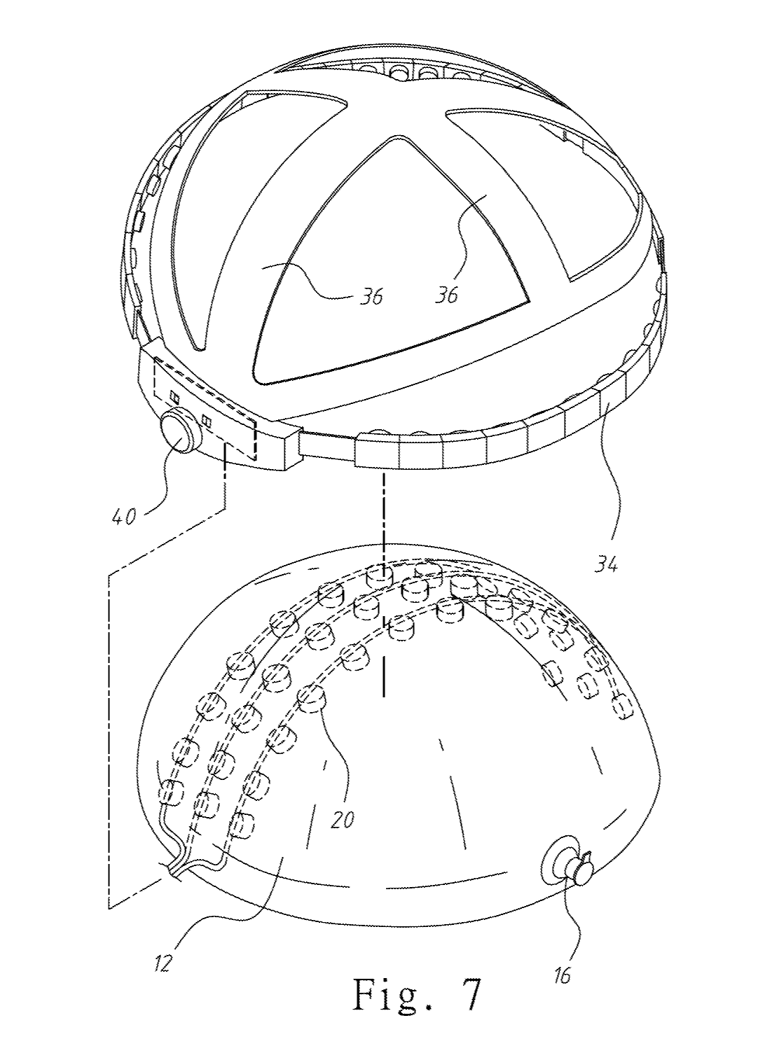

### DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts.

The embodiments described below are illustrated to demonstrate the technical contents and characteristics of the present invention and to enable the persons skilled in the art to understand, make, and use the present invention. However, it shall be noticed that, it is not intended to limit the scope of the present invention. Therefore, any equivalent modification or variation according to the spirit of the present invention is to be also included within the scope of the present invention.

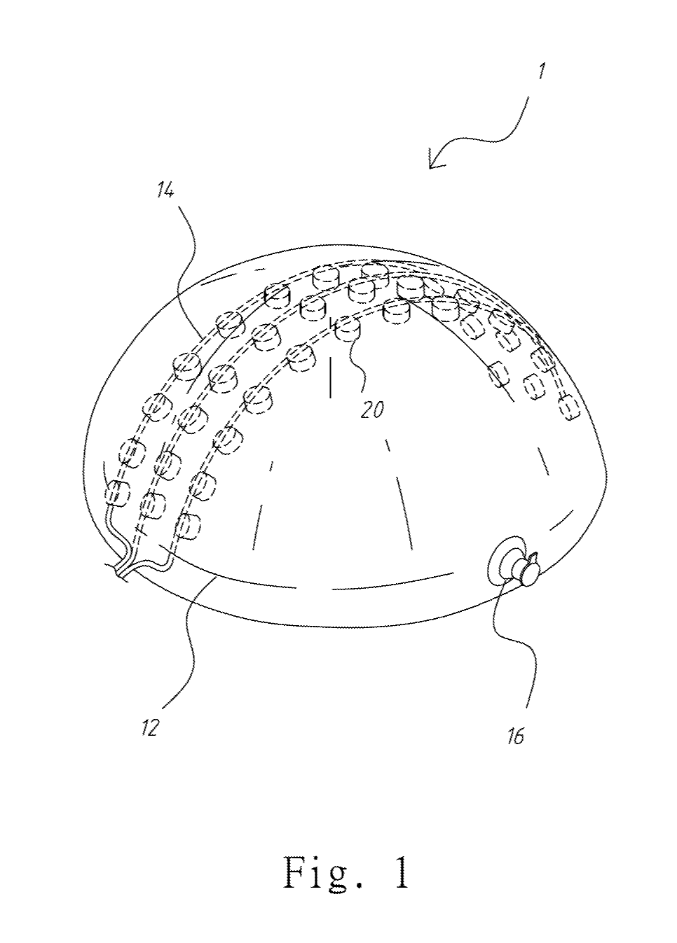

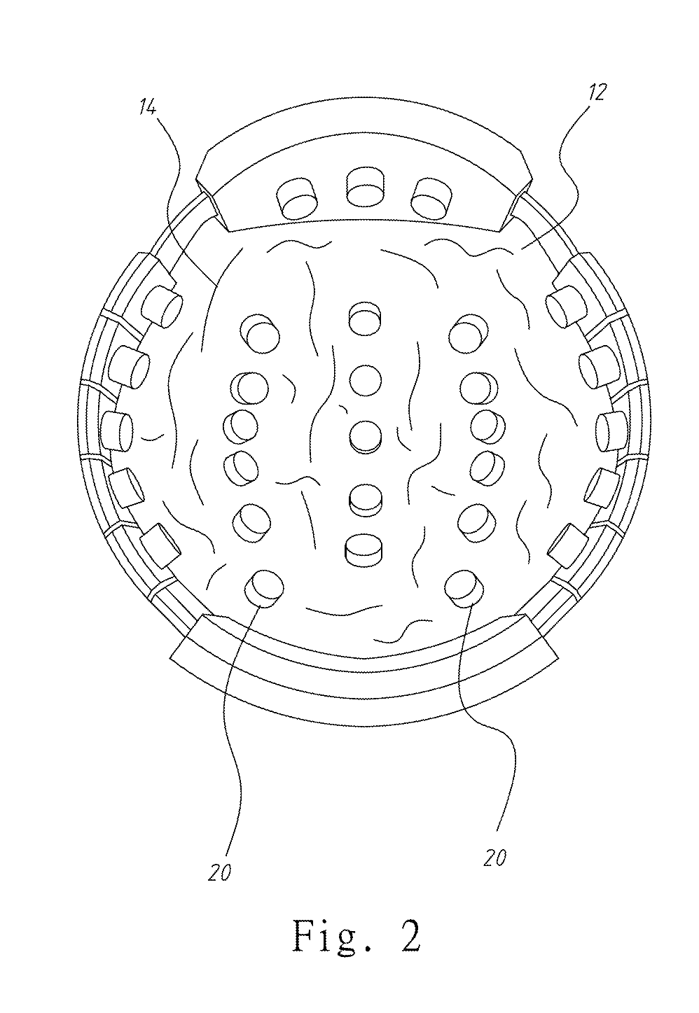

Please refer to FIG. 1 and FIG. 2, which respectively shows a three-dimensional schematic drawing and an inner drawing of the inflatable electroencephalogram measuring device in accordance with one embodiment of the present invention. As shown in FIG. 1 and FIG. 2, the inflatable electroencephalogram measuring device 1 of the present invention is a kind of EEG measuring device which is utilized for measuring a testee's EEG signals when being inflated. In this embodiment, the proposed inflatable electroencephalogram measuring device 1 is an independent air bag designed as a helmet shape for the testee to wear over his or her head, and there is one or more than one measuring electrode disposed on the inner side of the air bag in order to make contact with the testee's head. Therefore, by inflating the air bag, it oppresses the measuring electrode to effectively contact with the testee's head and to measure EEG signals of the testee. According to various embodiments of the present invention, the measuring electrode can be either directly oppressed or indirectly oppressed by inflation of the air bag. The present invention is not limited thereto.

### Detailed Descriptions Provided Below Show Explanation

According to a first embodiment of the present invention, the inflatable electroencephalogram measuring device 1 comprises an inflatable helmet and at least one measuring electrode 20. The inflatable helmet is suitable for the testee to wear over his or her head, and the inflatable helmet comprises an inflatable body 12 and a plurality of supporting strips 14, which are disposed on an inner side of the inflatable body 12. According to the embodiment of the present invention, the inflatable helmet further comprises an inlet 16, which is configured on at least one side of the inflatable body 12. Therefore, the inflation toward the inflatable body 12 can be done through air being input to the inlet 16. In other words, when an user blows air into the inlet 16, the inflatable body 12 gradually expands its volume. Furthermore, the number of the measuring electrode 20 of the present invention can be either one or more than one. Besides, according to the present invention, the proposed measuring electrode 20 can be adopted as using brush-electrode, which employs probes made of biomedical conductive materials and measures EEG signals when those probes effectively contact with the testee's skin. Alternatively, according to other embodiments of the present invention, the measuring electrode 20 may also be implemented by using the wet electrode or dry electrode which is commonly used for now. Since the measuring electrode 20 is disposed on the supporting strips 14, the measuring electrode 20 is induced to move closed to and make contact with the testee's head when the inflatable body 12 is being inflated. As a result, the EEG signals of the testee are successfully measured and recorded.

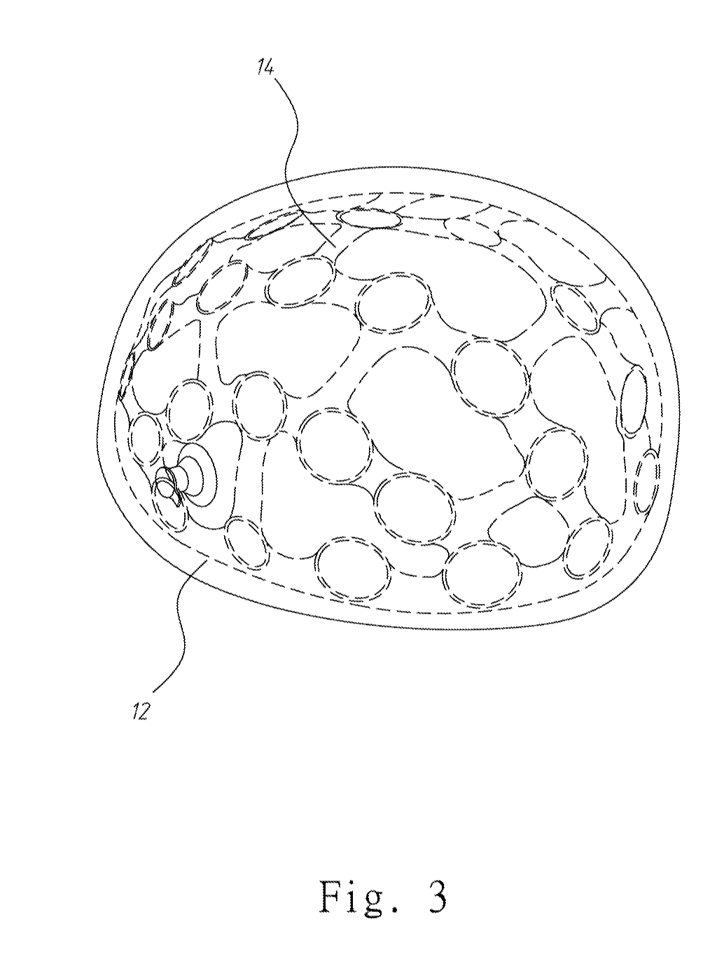

In another aspect, the inflatable electroencephalogram measuring device of the present invention further comprises a pressure measuring device, which is designed for controlling the pressure of the air bag (i.e. the inflatable body 12) within a safe range in case of damaging the testee's head. Also, when considering the inflatable body 12, the flow inside the air bag is designed to be asymmetric so as to make up where the measuring electrode is in loose contact with the testee's head, and to maintain where it is in good contact with the head as well. According to the embodiment of the present invention as shown in FIG. 3, the inflatable body 12 is made of one-piece structure, in which the inflatable body 12 is an air bag, disposed outside the supporting strips 14. As shown in the figure, the circle spots formed within the supporting strips 14 are where the measuring electrodes are configured to. As a result, when the inflatable body 12 is inflated, it oppresses the measuring electrodes to make contact with the testee's head so as to measure EEG signals of the testee. On the other hand, according to another embodiment of the present invention as shown in FIG.

4, the inflatable body 12 may also comprise one or more than one chamber 12a. That is to say, the inflatable body 12 of the present invention may further be designed in various deformation, for example, I-chamber, either based on different shape of testee's head or on different positions where the measuring electrode are located. In addition, when being inflated, the deformation of the inflatable body can be extended inwardly, outwardly, or both inwardly and outwardly at the same time. The present invention is apparent not limited thereto.

Furthermore, in order to make the proposed inflatable electroencephalogram measuring device itself much more delicate and soft and to be in better contact with the testee's head, the supporting strips 14 of the present invention are made of flexible materials, such as C-shape plastic or elastic plastics. As such, as shown in FIG. 2, each supporting strip 14 is believed to be bendable. As s result, by bending the supporting strips 14, it absorbs the stress generated by the moving measuring electrode 20. And thus, the testee may enjoy better comfort than ever when wearing the measuring device. According to one embodiment of the present invention, the measuring electrode 20 can be further connected to a printed circuit board (PCB) through a plurality of wires, such that the measured EEG signals detected by the measuring electrode 20 can be transmitted to the printed circuit board for further analyses.

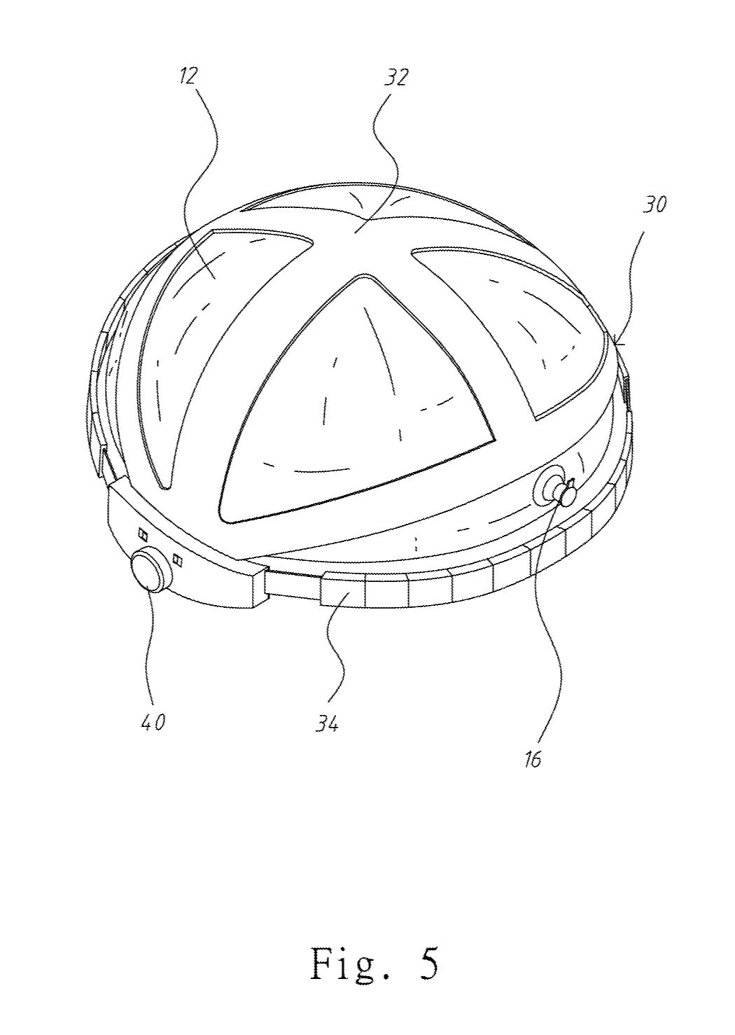

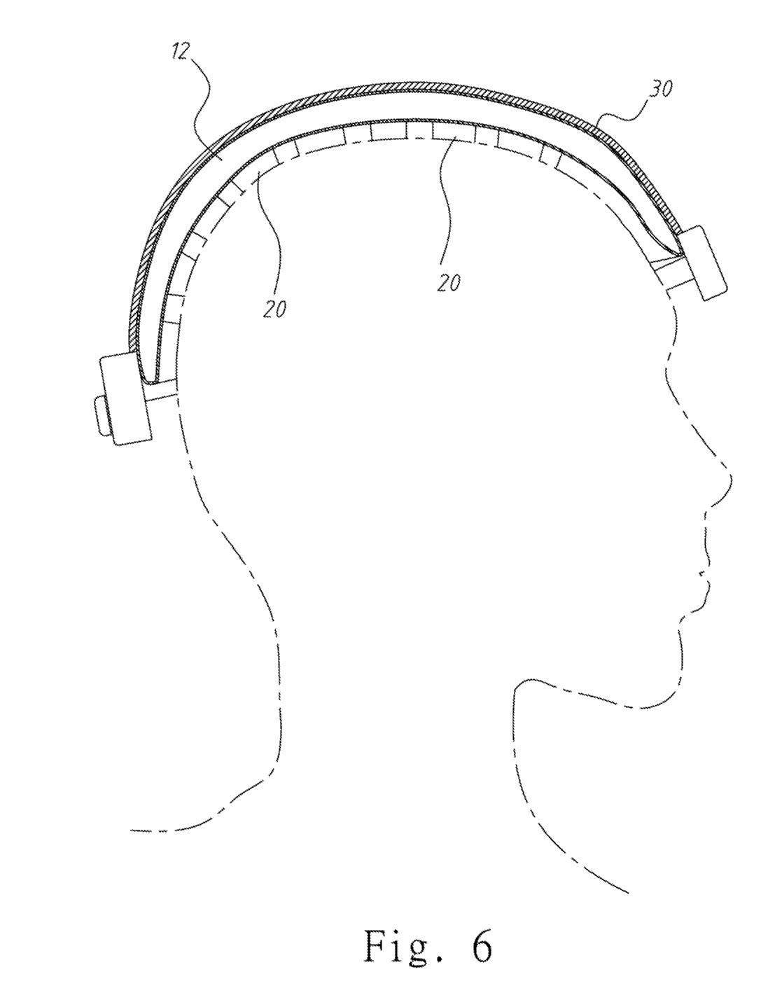

Still in another embodiment of the present invention, the inflatable helmet can also be designed as an air bag embedded in a hard case. Under such circumstance, the proposed inflatable electroencephalogram measuring device further comprises a housing 30 as shown in FIG. 5, and the inflatable helmet can be disposed in the inner space of the housing 30 for the testee to wear and measure the EEG signals. Therefore, in such embodiment, the inflatable body 12 is considered as an air bag embedded between the housing 30 and the supporting strips 14. FIG. 6 shows a schematic drawing of the testee wearing the housing 30 in order to measure EEG signals. As shown, after the testee wears the housing 30 and the embedded air bag is inflated, it also oppresses and induces the measuring electrode 20 to be in contact with the testee's head so as to measure EEG signals. In details, the housing 30 in the embodiment further comprises an upper cover 32 and a lower hoop 34, wherein the upper cover 32 connects the lower hoop 34 and extends upwardly from the lower hoop 34. Since the inflatable body 12 expands its volume when being inflated, the upper cover 32 may further comprise at least two fixing strips 36 which make across to each other so as to form some exposed spare room for absorbing the expanding volume of the inflatable body 12 when being inflated.

Moreover, an adjustment element 40, for example, a whirl button can be further disposed on the lower hoop 34 to help the measuring device of the present invention fit the testee better. Under such circumstance, when the testee wears the housing 30 and the lower hoop 34 surrounds the head, the adjustment element 40 can be controlled to make the housing 30 tighter or looser so as to fit the head size of the test much more precisely.

As a result, to sum up, the present invention indeed provides a novel and inventive inflatable electroencephalogram measuring device which has never been seen or proposed ever before when compared to the prior arts. By employing the proposed inflatable helmet being inflated, it is able to oppress and induce the measuring electrode connected thereto to make better contact with the testee's head. As a result, the conventional issues that the typical brave-waves measuring devices are mostly limited to a certain head size are successfully solved.

Moreover, since the material of the inflatable helmet is quite easy to obtain, for example, common plastic ball or rubber ball, thus the production and fabrication cost of the present invention can be further reduced even if being made in mass production. Therefore, it is believed that the inflatable electroencephalogram measuring device of the present invention is meant to be major appliance for further biomedical developments.

Furthermore, the inflatable electroencephalogram measuring device of the present invention is designed to be adjustable according to different head size of different testee. No matter what size, shape, cave or protuberance the testee' head is having, the inflatable helmet is able to be adjusted in order to fit the testee's head much more accurately and precisely. As a result, it is obvious and apparent that the inflatable electroencephalogram measuring device of the present invention is able to be widely used for researches and industries developed in the future, thereby being advantageous of better competitiveness and potential growing in a prospective aspect.

It will be apparent to those skilled in the art that various modifications and variations can be made to the present invention without departing from the scope or spirit of the invention. In view of the foregoing, it is intended that the present invention cover modifications and variations of this invention provided they fall within the scope of the invention and its equivalent.

What is claimed is:

1. An inflatable electroencephalogram measuring device for measuring a testee's EEG signals, comprising:
an inflatable helmet for said testee to wear over his or her head, comprising an inflatable body and a plurality of supporting strips which are disposed on an inner side of said inflatable body; and
at least one measuring electrode disposed on said plurality of supporting strips, such that when said inflatable helmet is inflated, said measuring electrode is induced to make contact with said head to measure said testee's EEG signals.

2. The inflatable electroencephalogram measuring device of claim 1, wherein said inflatable helmet further comprises an inlet, which is disposed on one side of said inflatable body, and air can be blown into said inflatable body through said inlet for inflating said inflatable body.

3. The inflatable electroencephalogram measuring device of claim 1, further comprising a plurality of wires and a printed circuit board, wherein said plurality of wires are connected between said measuring electrode and said printed circuit board, and said testee's EEG signals are transmitted to said printed circuit board through said plurality of wires for further analyses.

4. The inflatable electroencephalogram measuring device of claim 1, wherein said measuring electrode is brush-electrode, dry electrode or wet electrode.

5. The inflatable electroencephalogram measuring device of claim 1, wherein each of said plurality of supporting strips is bendable so as to absorb stress generated by said moving measuring electrode.

6. The inflatable electroencephalogram measuring device of claim 1, wherein each of said plurality of supporting strips is made of flexible material.

7. The inflatable electroencephalogram measuring device of claim 1, further comprising a housing, and said inflatable helmet being disposed in an inner space of said housing for said testee to wear so as to measure said EEG signals.

8. The inflatable electroencephalogram measuring device of claim 7, wherein said housing further comprises an upper cover and a lower hoop, said upper cover connects said lower hoop and extends upwardly from said lower hoop, said lower hoop surrounds said testee's head, and said inflatable helmet is disposed on an inner side of said upper cover.

9. The inflatable electroencephalogram measuring device of claim 8, wherein said upper cover further comprises at least two fixing strips which make across to each other, and said fixing strips are commonly connected to said lower hoop.

10. The inflatable electroencephalogram measuring device of claim 8, wherein said lower hoop further comprises an adjustment element for controlling said housing tighter or looser.

11. The inflatable electroencephalogram measuring device of claim 1, wherein flow inside said inflatable body is designed to be asymmetric.

12. The inflatable electroencephalogram measuring device of claim 1, further comprising a pressure measuring device, which is designed for controlling pressure of said inflatable body within a safe range in case of damaging said testee's head.

13. The inflatable electroencephalogram measuring device of claim 1, wherein said inflatable body is a one-piece structure or comprises at least one chamber.

14. The inflatable electroencephalogram measuring device of claim 13, wherein inflation of said chamber can be extended inwardly, outwardly, or both inwardly and outwardly equally.

15. The inflatable electroencephalogram measuring device of claim 13, wherein deformation of said chamber can be designed either based on different shape of said testee's head or on different positions where said measuring electrode is located.
